(19) United States
(12) Patent Application Publication
(10) Pub. No.: US 2012/0164739 A1
(43) Pub. Date: Jun. 28, 2012

(54) DUAL HEAT STABILIZED POLYMER SENSOR FILMS

(75) Inventors: Bingzhi Chen, Harleysville, PA (US); Scott Martell Boyette, New Hope, PA (US); Janine Clemens, Doylestown, PA (US); Vidyasankar Sundaresan, Lansdale, PA (US); Hong Xu, Shanghai (CN)

(73) Assignee: GENERAL ELECTRIC COMPANY, Schenectady, NY (US)

(21) Appl. No.: 12/977,558

(22) Filed: Dec. 23, 2010

(51) Int. Cl. G01N 33/20

(52) U.S. Cl. 436/79

(57) ABSTRACT

Methods are provided for improving the stability and shelf life of film sensors. The film sensors include a combination of polymeric matrix film material, analyte indicator, and solvent which has been heated in a first heat treatment application to volatilize most of the solvent and to solidify the analyte indicator to form the film sensor. The improvement comprises subjecting the film sensor to a second heat treatment to set the film and improve stability and shelf life.

### FIELD OF INVENTION

The application pertains to processes for treating polymer based film sensors to increase stability and shelf life.

### BACKGROUND OF THE INVENTION

Sensor films are disclosed in U.S. Pat. No. 7,807,473 of the type wherein a polymeric substrate or film is provided with an indicator composition thereon that is capable of measuring a variety of analyte types and concentrations by a measured change in the optical properties of the indicator such as changes in elastic or inelastic scattering, absorption, luminescence intensity, luminescence lifetime, or polarization state. The main components include a chemically sensitive reagent (i.e., the indicator), a polymeric matrix, auxiliary minor additives and a common solvent or solvent mixture. The entire content of U.S. Pat. No. 7,807,473 is incorporated by reference herein.

Sensor life or shelf life is one of the biggest product concerns related to commercial viability of the sensor. In this respect, shelf lives of six months or more are needed for commercially distributed sensors. It is also desirable for film sensors to be stored and shipped without requiring special insulation or temperature and/or humidity control. Special handling can complicate distribution methods and dramatically increase final use cost. However, even with progress on commercialization of film sensors, the shelf life is still a key issue. Many film sensors have to be stored under stringent conditions or will expire within a short time. The reasons for short shelf lives vary, and can be from physical property changes that cause performance drift such as wetability of the film, permeability of the film, resistance, and component phase change, changes in chemical properties, including, component decomposition, and polymer substrate aging. These are just some of the causes for sensor film performance loss over time.

Accordingly, there is a need in the art for methods for increasing stability and shelf life of polymer based film sensors.

### SUMMARY OF THE INVENTION

In one exemplary embodiment, a method is disclosed for treating a film sensor of the type including a combination of polymeric matrix film material, analyte indicator, and solvent. This combination has already been treated via a first heat application to volatize most of the solvent and to solidify the analyte indicator to form a film sensor. In accordance with one aspect of the invention, the improvement comprises subjecting this film sensor to a second heat treatment to set the film and improve stability and shelf life. The second heating may comprise heating of the film sensor at a temperature of from about 50° C. to about 150° C. for a period of 1-3 days. More preferably, the second heating comprises heating the film sensor at a temperature of about 70° C. to 130° C. for a period of about 5 minutes to 2 days. Most preferably, the second reheating step comprises reheating of the film for a period of about 5 minutes to 10 hours at a temperature of about 70° C. to 130° C.

In another exemplary embodiment, the film sensor is adapted to determine calcium concentration in an aqueous sample. In this case, the polymer matrix comprises poly(2-hydroxyethylmethacrylate) (pHEMA) hydrogel and the analyte indicator comprises chlorophosphonazo III.

In another embodiment, the film sensor is adapted for determining total hardness in an aqueous sample with the polymeric matrix comprising (pHEMA) hydrogel and the analyte indicator comprising methylthymol blue.

In yet another aspect, the film sensor is adapted for determining magnesium concentration in an aqueous sample. The polymeric matrix comprises (pHEMA) hydrogel, and the analyte indicator comprises Eriochrome Black T.

The invention will be further described in conjunction with the following detailed description and appended drawings.

### BRIEF DESCRIPTION OF THE DRAWINGS

FIG. 1 is a graph showing the results of an accelerated aging study completed on a film sensor that has not been treated with a post treatment process;

FIG. 2 is a graph showing the results of a long term room temperature stability study completed on the film sensor of FIG. 1;

FIG. 3 is a graph showing the results of an accelerated aging study completed on a high temperature dried film sensor that has not been treated with a post treatment process;

FIG. 4 is a graph showing the results of an accelerated aging study completed on a film sensor treated with a post treatment heating step in accordance with the invention;

FIG. 5 is a graph showing the results of a long term room temperature stability study on a film sensor treated in accordance with the invention;

FIG. 6 is a graph showing the results of a long term stability study on another film sensor treated in accordance with the invention;

FIG. 7 is a graph showing the results of a long term stability study on another film sensor treated in accordance with the invention;

FIG. 8 is a graph showing the results of a long term stability study on a comparative film sensor that had not been post treated in accordance with the invention;

FIG. 9 is a graph showing the results of an accelerated aging test conducted on a film sensor that has been post treated in accordance with the invention; and

FIG. 10 is a graph showing the specificity for Mg film sensor based on Eriochrome Black T.

### DETAILED DESCRIPTION

Methods for making the film sensors include flow coating, dip-coating, screen-printing, draw coating, spray coating, Gravure coating, ink jet printing, casting, and drop formation. Depending on the formula (Ink) properties, different processes for solidifying sensor formulations inks will be applied, such as heating, freeze drying, UV-curing, room temperature evaporation, air blowing, vacuum desiccation, and others. The solidifying process varies and can take from seconds, to tens of minutes, to hours. Depending on the curing process utilized, some of the chemical and physical properties may not be not fully cured and can be trapped at a non-equilibrium state. Once the film has been partially cured, the trapped unstable chemical and physical properties will keep changing towards their thermodynamically stable equilibrium. This can be a very slow process. These slow chemical and physical properties changes are some of the key factors causing film sensor performance drift and aging.

Efforts to stabilize film sensors during the solidifying process have many limitations. Some of the reasons are: 1) the stabilizing process might significantly slow down the production process and decrease productivity; 2) techniques like flow coating need a much longer oven for extended heating times; slowing down the coating will decrease the productivity and raise the possibility of a coating defect, such as created by coater mechanical instability that results in web chatter; 3) over drying (heating) to accelerate the property change might cause sensor ingredients to decompose when dried too quickly or at extreme temperatures; and 4) film sensors produced on polymer substrates, such as PVC, PET will start to lose physical integrity at high temperatures and at extended heating times.

In one exemplary embodiment of the invention, a post-treatment method for sensor film stabilization has been developed and involves re-heating of the film sensor. The components for film sensors include solvent, polymer as film sensor substrate, indicator (colorimetric, fluorescent, magnetic, etc.) and other auxiliary reagents, such as surfactants, plasticizer, inert color reference standards, etc. The components are deposited on a solid substrate through techniques like flow coating, screen printing, and these are followed by solidifying the film in ovens with varying time from seconds to minutes. Once most of the solvent has been volatilized, and the coated ink has solidified to a film, a number of simple post-treatment approaches have been developed. First, the film can be re-dried (i.e., an additional heating step) by use of the same drying process after the formula ink (i.e., the indicator) has dried. For example, if flow coating was used to form the film sensor, a roll of the so-formed film can be re-wound and re-dried (heated) through the oven system. Additionally, the film can simply be re-heated right before it is needed for its analytical, testing function. Thirdly, the finished sensor product can be re-heated.

This re-drying step, or as sometimes referred to herein as re-heating can be conducted at a variety of different temperatures and for a variety of different durations. The re-drying step can be conducted at a higher, the same, or lower temperature than the original drying temperature. Also, the re-drying temperature could proceed along a varying time/temperature gradient. The sensor film could be dried in stages, such as by an initial first stage drying at a given time and temperature followed by a cooling period and then it could be dried again at a second temperature to set the film and impart greater stability and longer shelf life.

Sensor films require uniform, defect free coatings. Sometimes, raising the temperature in a drying process too quickly results in physical defects commonly referred to as orange peel, onion skin, or scaling. Sometimes, the defects may be seen in the form of “mixed film domains” wherein, for instance, an inner film portion contains a greater solvent and/or reagent content than the top or surface layer. The rate at which a film is dried is also a factor. Sometimes, a slower temperature ramp may prove impractical in imparting the desired stability. The dual pass process in accordance with the invention allows film materials to undergo additional physical and chemical changes that allow the second pass to better lock in desired sensor film properties and impart greater long-term stability.

During the re-heating post-treatment process, a number of thermodynamic unstable chemical and physical properties can be accelerated toward a stable state, thus the unstable factors can be eliminated and the film sensors will be pre-stabilized so that they have longer shelf lives. Depending on the film sensor compositions, these properties could be, but are not limited to, a change during the re-heating process that may cause 1) wetability changes in the film sensor; 2) permeability of the film sensor; 3) micelle formation or breaking if a surfactant is in the film sensor; 4) dehydration if a hydrated chemical is in the formulation; 5) homogenization of film sensor if a discrete micro phase exists is in the film sensor or vice verse; 6) crystal morphology transfer; 7) cross-linking of polymer substrates; 8) interaction between ion pairs, such as cationic dyes with quaternary amine surfactant; 9) unstable impurity in dyes (most of dyes of less than 95% purity) decomposed to a point will not interfere the detection anymore; 10) removal of residual solvent; and 11) change in polymer conformation that locks reagents and minimizes diffusion and chemical interactions.

### EXAMPLES

The invention will be further described in conjunction with the following illustrative examples.

### Example 1

The post-treatment, re-heating process can be carried out right after the formula (Ink) is deposited on the carrier substrate.

The calcium specific sensor formula includes dye chlorophosphonazo III as indicator, pHEMA as hydrogel, Zeph as dynamic range modifier, phthalate as buffer, Dowanol DM, Dowanol PM, and water as solvents. The formula is flow coated on PET substrate and dried at 100° C. for 10 minutes, or 130° C. for 10-minutes. The solvent residual was checked and was less than 0.5% using film dissolution and measuring residual solvent content measured by high-pressure liquid chromatography. These no-post-treatment films were identified as “mono-pass calcium film sensor”. In accordance with the invention, the film 100° C. “mono-pass calcium film sensor” was re-heated through the same drying oven system at 127° C. for 10 minutes, and the post-treated film was identified as “double-pass calcium film sensor”. To compare the stability of both sensor film types, accelerated aging studies and long-term room temperature stability checks were performed. The accelerated aging study is performed at 70° C. for different times (0-24 hours) to create an aggressive environment that will force the film to its ultimate composition/state. This accelerated process is used to mimic longer-term room temperature studies that take too long to be of practical use, and the comparison of film sensor performance at 70° C. and different times was used to determine if the film is stable and has a longer shelf life.

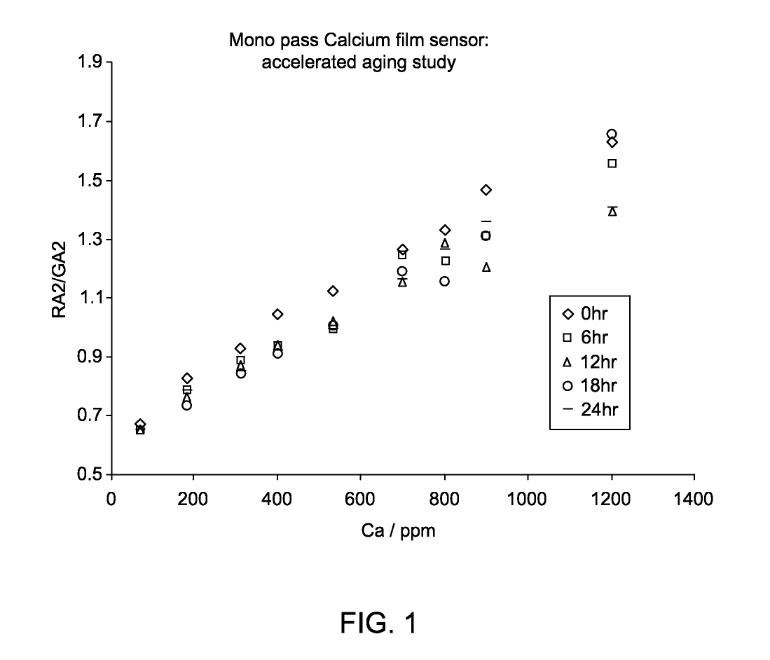

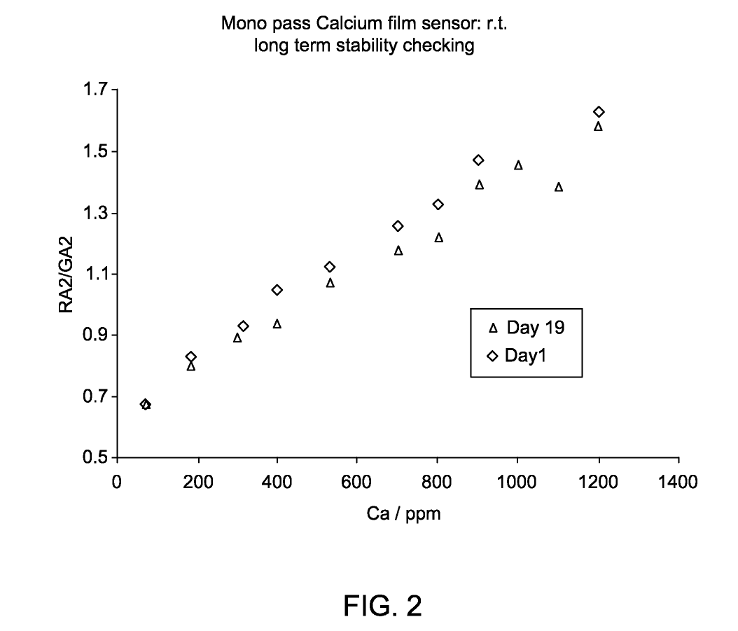

For the mono-pass calcium film sensor dried at 100° C., the accelerated aging study and long-term room temperature stability checks are shown in FIG. 1 and FIG. 2 respectively. In FIG. 1, it is clear that the accelerated aging at 70° C. causes the Ca performance curve to drift over time, which is unacceptable for practical applications. In FIG. 2, the room temperature, long-term stability check shows the sensor performance becomes significantly depressed, and lasts just three weeks under room temperature storage. This result is consistent with the accelerated aging study.

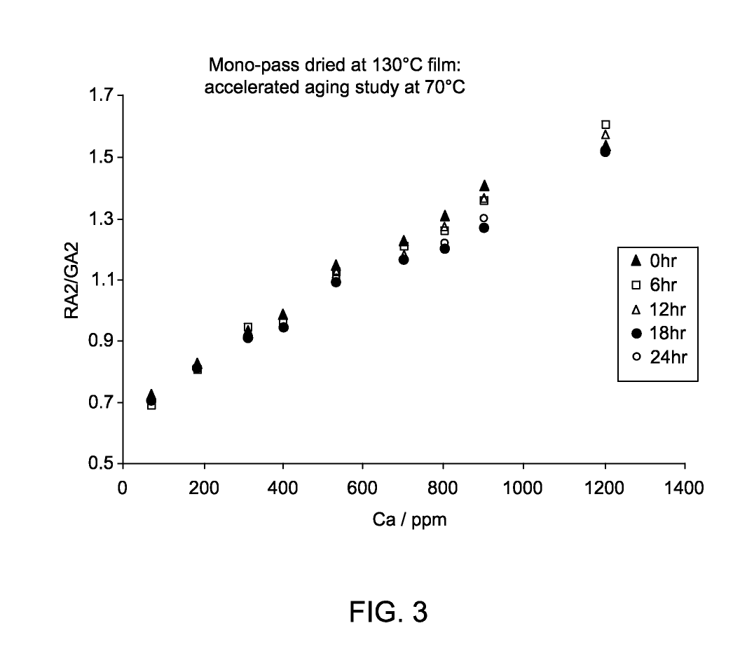

The mono-pass calcium film sensor dried at 130° C., the accelerated aging study and long-term room temperature stability checks are shown in FIG. 3. This figure shows that just elevated temperature drying is insufficient to impart longer-term film sensor stability. Accelerated aging studies on the mono-pass calcium film sensor dried at 130° C. show that the stability has improved when compared to the mono-pass calcium film sensor dried at 100° C., but is still insufficient to induce the desired long-term stability.

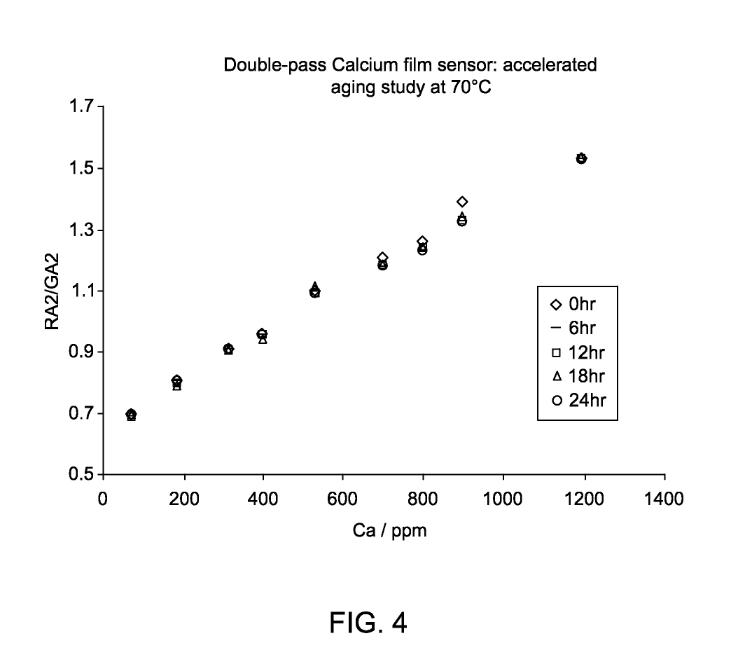

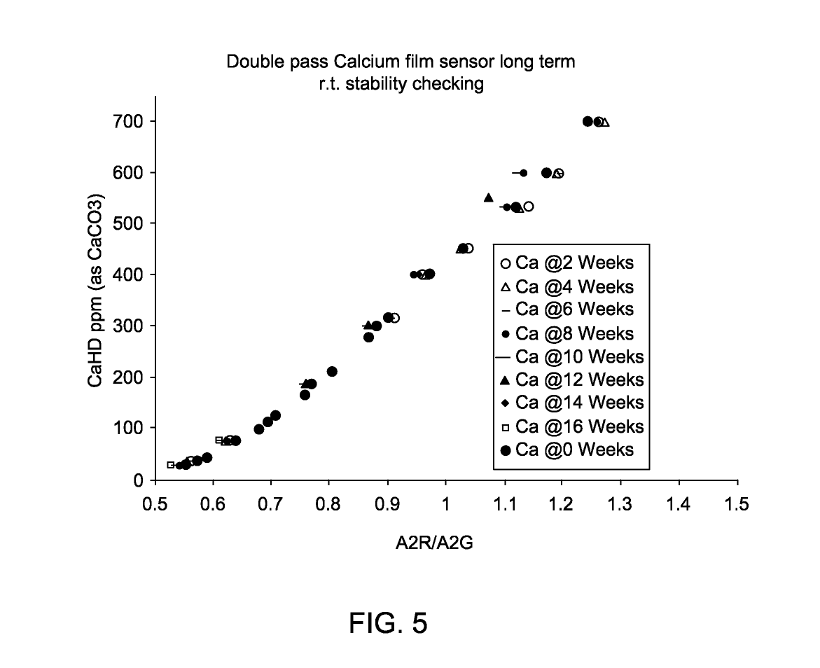

For the double-pass calcium film sensor, the film stability was also studied through accelerated aging test and long-term room temperature stability checking as shown in FIGS. 4 and 5 respectively. The first pass drying is mainly for solidifying the deposited formula (Ink) onto a substrate, such as PET. The temperature can be from 50° C. to 130° C., but is not limited to any specific combination of time and temperature. The resulting film contains little residual solvent and is considered a completely dry film sensor. The second pass drying could also range from 70° C. to 130° C. with different times. In this list, the reheating conditions were 100° C. for 10 minutes and 127° C. for 10 minutes. The accelerated aging study shows that after 24 hours heating at 70° C., the double-pass calcium film sensor performance didn't change. Based on our accelerated aging model, the predicted shelf life should be much longer than six months. The long-term room temperature stability check of the double-pass calcium film sensor lasted for 14 weeks and is shown in FIG. 5. The performance remained very stable without any significant performance loss or drift over 14 weeks. During the coating and double-pass drying process, the interaction of Zeph/phosphoric groups is suspected to have changed. Once the sensor is deposited on substrate (PET film in this case), and double-pass dried the film is cooled to room temperature. Thus, the thermodynamic stable state of the film can be trapped. In contrast, the thermodynamically unstable interaction might take day/months, or even years to reach thermodynamic stability with just a single-pass drying, which could cause performance drift over the time. The post-treatment, re-heating, accelerated the thermodynamic favorable process. With the right combination of temperature time, the interaction of sensor components can reach a thermodynamically stable state. This will eliminate or minimize performance drift and stabilize the film. Additional physical properties can also reach thermodynamic stability during the post-heating process, such as but not limited to, wetability, surface tension, and permeability.

### Example 2

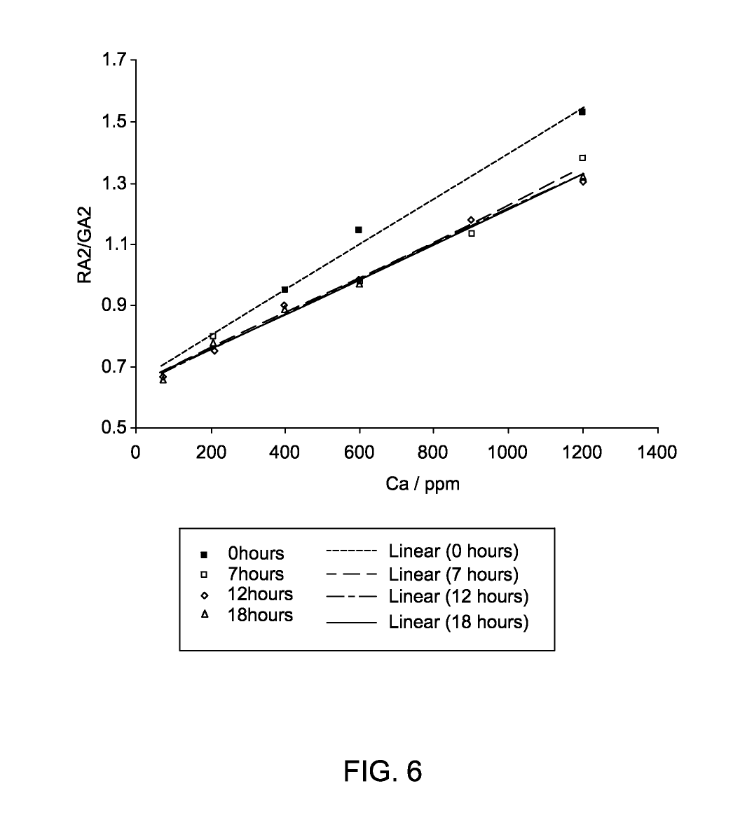

The mono-pass dried film sensor from a flow coating process can also be post-treated. One example is a mono-pass dried film that was re-heated at 70° C. for 7 hours in an oven as shown in FIG. 6. After 7 hours at 70° C., the performance was stabilized. The evidence for stability is that reheating for 70° C. for 12 hours and 18 hours did not further change the sensor film performance, although the original performance was not fully recaptured with the lower temperature second-pass drying. This off-coating system re-heating could significantly increase the productivity of film sensor because it did not use the flow-coating system for re-heating. The film sensor can be re-heated in a roll, after slitting, or after the sensor system or array has been assembled. The sensor film stability can be imparted in different stages of the sensor making process, and this provides additional flexibility for optimizing the sensor system production process.

### Example 3

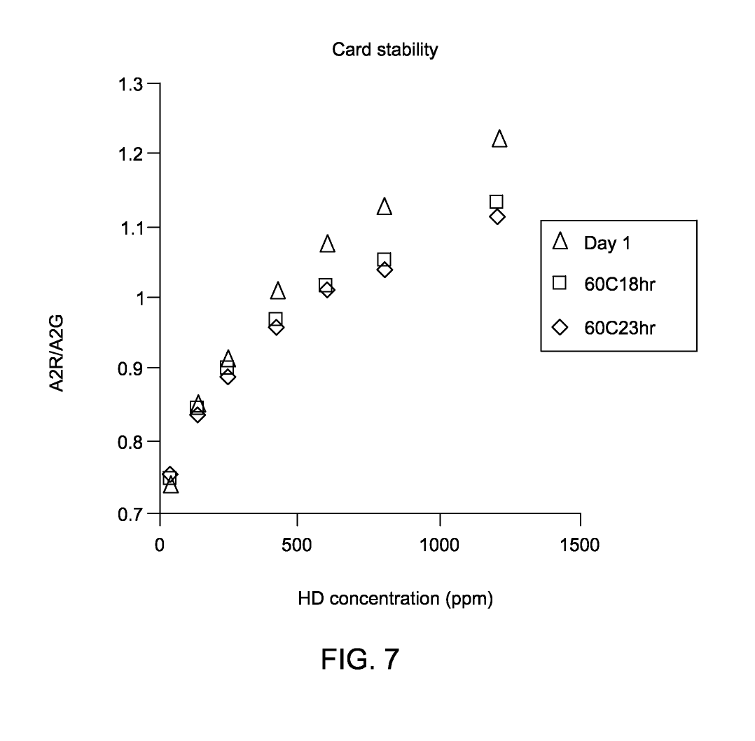

A total-hardness sensor film sensor was produced using a similar double pass process. The total hardness sensor formula includes MTB (methylthymol blue) as indicator, Zeph as immobilizer and dynamic range modifier, TEA (triethanolamine) as buffer, pHEMA as hydrogel polymer support, Dowanol DM and Dowanol PM as solvents. As shown in FIG. 7, the total hardness film performance was not stable by mono-pass drying at 80° C. for 10 minutes because accelerated aging study at 60° C. for 18 hours shows a significantly different calibration curve. However, after re-heating for 18, 23, or more hours at 60° C., the film was stabilized and showed no further performance loss after been dried at 60° C. for extended times. This shows that the re-heating (post-heating,) is a useful approach to stabilize the performance of film sensors and obtain a longer shelf life.

### Example 4

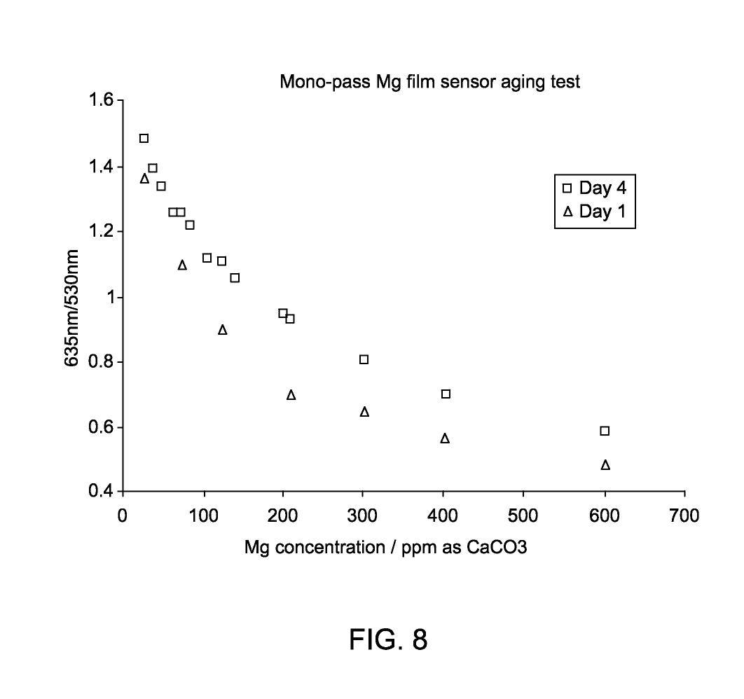

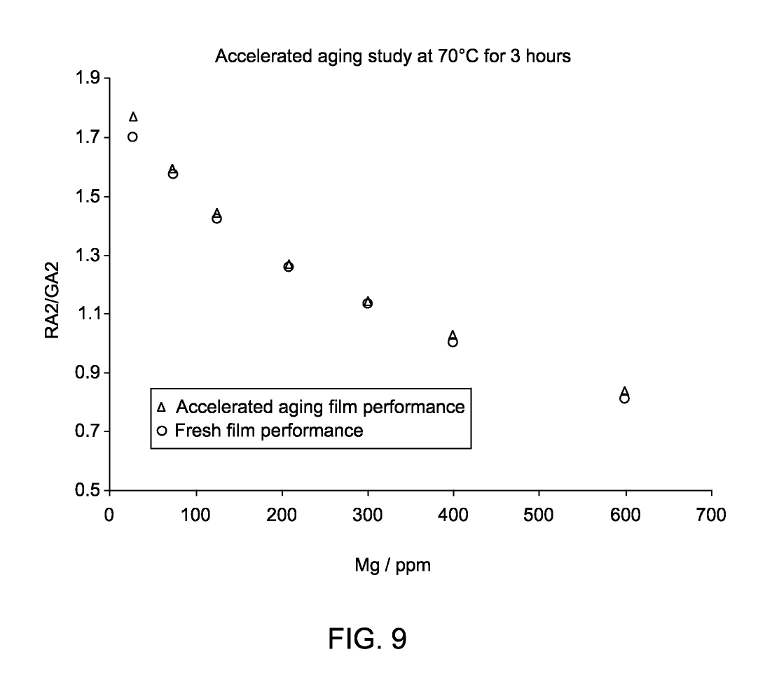

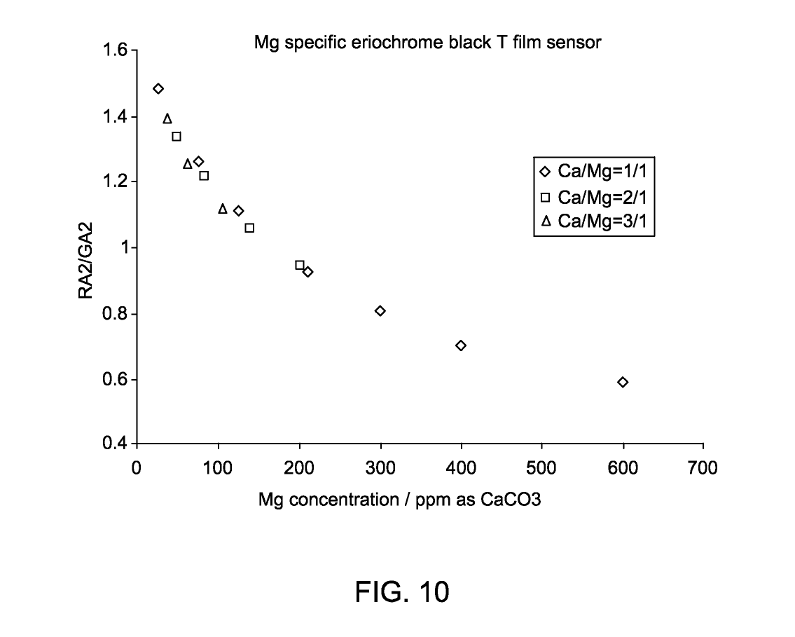

A magnesium specific film sensor shows that re-heating (post-heating,) is a useful technique to stabilize film sensor performance. The magnesium sensor formula includes Eriochrome Black T as an indicator, TBAB (tetrabutylammonium bromide) as an immobilizer and dynamic range modifier, TEA (triethanolamine) as a buffer, pHEMA hydrogel and Dowanol DM/PM as solvents. The formula (Ink) was flow coated and dried at 70° C. for 10 minutes. This is referred to as mono-pass Mg film sensor. The formula can also be flow coated and dried at 100° C. for 10 minutes, followed by second pass at 127° C. for 10 minutes. In FIG. 8, the mono-pass Mg film sensor shows significant performance drift after only 4 days at room temperature storage. While, in FIG. 9, the double pass Mg film sensor shows very stable performance when an accelerated aging study was carried out at 70° C. for 3 hours. This magnesium specific film sensor is also novel in that Eriochrome Black T indicator is not Mg-specific in water phase testing, and will respond to both Ca and Mg, but when incorporated into this specific solid film sensor becomes Mg-specific. The film was thus tested by standard solutions with varying Mg concentrations and varying ratio of calcium over magnesium. The FIG. 10 shows the calibration curve. It is obviously that the dynamic range of the sensor is from 25 ppm up to 600 ppm. The response curve is independent on the Ca/Mg ratio, which proves the Mg specific feature of the film sensor. This demonstrates that the performance of film sensors can be significantly different from chemistries applied in solution or water-based applications.

Sensors of the type that may employ the double pass or double heating step of the invention in order to improve stability and shelf life may for example be either Ca or Mg specific or the sensors may be adapted to detect both of these commonly encountered water-borne ions.

Embodiments of self-contained calcium ion specific and magnesium ion specific solid film sensors described herein contain at least an analyte specific reagent, a pH modifier and a sensitivity (dynamic range) modifier. The analyte-specific reagent combined with optimized pH modifier and sensitivity modifier can provide calcium ion specific and magnesium ion specific detection capabilities. The physical and chemical properties of the transparent sensor films change as a result of contact of the film sensors with aqueous samples having different concentrations of calcium and magnesium ions. The response signal can be acquired and analyzed at minute scale level. Self-contained calcium or magnesium specific film sensors have the advantage that no post-addition reagents, pre-concentration or dilution are required to determine the Ca (or Mg) concentration. The analysis of Ca (or Mg) in a given sample needs only a minimal number of procedural steps. Moreover, the sensitivity (dynamic range) of the self-contained film sensor can be tuned by different types and amounts of sensitivity modifiers, such as quaternary amines. The Ca or Mg concentration in a test sample can be quantified using a calibration curve generated by testing samples with known Ca or Mg concentrations.

Initially, the selection of an analyte-specific reagent must be made for a Ca/Mg specific solid film sensor. Analyte-specific reagents are compounds that exclusively respond to the analyte or preferably respond to the analyte over other co-existing interfering chemicals in the test sample. Analyte-specific reagents may include metal complexes or salts, organic and inorganic dyes, advanced functional polymers, etc. The reagent should be exclusively respond to calcium or magnesium ions. The analyte-specific reagent can also be more selective to calcium over magnesium or vice versa. Upon contact with the analyte, the analyte-specific reagent will exhibit a detectable change in a chemical or physical property useful for the identification of the analyte chemical and biological species. For example, optical property changes include absorption, luminescence, or reflectance, and these may be correlated with calcium ion concentration. Moreover, having charged functional groups allows for the formation of an ionic pair with the quaternary ammonium, with attendant liphophilic characteristics. This facilitates its solubility in inks and immobilization of the reagent on matrices.

In one aspect, the analyte-specific reagent used in the self-contained film sensor can be a dye. The dye is a chromogenic indicator. “Chromogenic” means that a characteristic of a chemical system whereby a detectable response is generated in response to an external stimulus. Thus, for example, an ionophore is chromogenic when it is capable of exhibiting a detectable response upon complexing with an ion, where the detectable response is not limited solely to change in color as defined below. “Detectable response” means a change in or appearance of a property in a system, which is capable of being perceived, either by direct observation or instrumentally, and which is a function of the presence of a specific ion in a test sample. Some examples of detectable responses are the change in or appearance of color, fluorescence, phosphorescence, reflectance, chemiluminescence, or infrared spectrum. Other examples of detectable responses may be the change in electrochemical properties, pH and nuclear magnetic resonance. Some examples of suitable dyes that may be employed in the analyte-specific reagents include, but not limited to, azo dyes, anthraquinone dyes, triphenylmethane dyes.

Generally, the self-contained calcium specific and magnesium specific solid film sensors include pH modifiers that serve as buffers and maintain the pH level of the sensor formulations at a constant pH, which is preferable for the sensing mechanism. The choice of pH modifiers depends upon the nature of the analyte-specific reagent used, but pH-modifiers many include acids, bases, or salts.

In one aspect, a self-contained calcium specific and magnesium specific solid film sensor can include bases as pH modifiers. Bases as pH modifier may include inorganic, organic, polymeric chemicals. The inorganic bases could be sodium hydroxide, potassium hydroxide, etc. The organic bases could be, but are not limited to primary, secondary and tertiary amines and quaternary ammonium hydroxide and their combinations. Examples include, but are not limited to, triethanolamine, diethanolamine, triethylamine, tributylamine, N,N-dimethylethanolamine, 3-methoxypropylamine, aminopropyldiethanolamine, bis(3-aminopropyl)ethylenediamine, butylamine, cyclohexylamine, dibutylamine, diethylenetriamine (DETA), dihexylamine, dimethylaminoethanol, dimethylaminopropylamine, ethanolamine, ethylenediamine, hexamethylenetetramine, N,N-diethylethanolamine, N,N. dimethylcyclohexylamine, tetraethylenepentamine, triethylene pentamine, tetramethylammonium hydroxide, tetrabutylammonium hydroxide, N,N,N,N′,N′,N′-hexabutylhexamethylenediammonium dihydroxide,
N,N,N,N′,N′,N′-hexabutylhexamethylenediammonium dihydroxide, N,N,N′,N′-tetraethyldiethylenetriamine, 1,1,4,7,10,10-hexamethyltriethylenetetramine, N,N,N′,N′-tetrakis(2-hydroxypropyl)ethylenediamine, tris[2-(isopropylamino)ethyl]amine, 3,3′-iminobis(N,N-dimethylpropylamine), diethylenetriamine. The polymeric amines could be, but are not limited to, poly(propylenediamine), polyethyleneimine with different molecular weight distribution. The optimum pH provided by bases pH-modifier can be achieved by pH adjustment with acids.

A myriad of acid pH modifiers may be provided as long as they are miscible with the sensor formulation and not volatile during the film drying process. The acid could include small organic molecules, or polymers having acid functional groups and the combination of them. The acid functional groups could be, but are not limited to, carboxylic acid, sulfonic acid, phosphoric acid and boric acid. Examples are p-toluenesulfonic acid, citric acid, phthalic acid. The optimum pH provided by the acid pH-modifier can be achieved through pH adjustment with bases.

Buffers could be used, even preferably used as pH-modifier in the self-contained calcium specific and magnesium specific film sensors. Buffers can not only provide more precise pH level control, but also provide abundant choices to cover wide pH ranges. Buffers could be inorganic buffers, organic buffers, and biological buffers. In one aspect, to provide a mild pH window, lots of biological buffers are very good candidates because of their extremely low volatility, less dependence on ionic strength, good solubility both in water and organic solvent based sensor formulation and readily commercial availability. Examples are, but not limited to, N,N-bis(2-hydroxyethyl)-2-aminoethanesulfonic acid (BES), N,N-bis(2-hydroxyethyl)glycine (BICINE), 4-(2-hydroxyethyppiperazine-1-ethanesulfonic acid (HEPES), N-[tris(hydroxymethyl)methyl]-3-aminopropanesulfonic acid (TAPS). The optimum pH provided by the buffers can be achieved through pH fine-tuning by acid and bases addition.

Film sensors (test strips) have been prepared by immobilizing dyes and other auxiliary reagents in matrices on solid supports. Reagents with sulfonic acid or carboxylic acid functional groups can be immobilized thorough anion exchange with immobilizing reagents (such as quaternary amine). However, it is necessary that the reagent retain its chelating or sequestering properties after being immobilized on the support. After ion exchange, the ion pair formed by negatively charged groups, such as sulfonic and carboxylic groups with positive charged functional groups such as quaternary amine is much more hydrophobic. The hydrophobicity of the ion pair is the key to making non-leachable film. Generally, immobilization reagents impart hydrophobic properties to the dyes, thus making the dyes non-leachable. The most widely accepted immobilization reagents are quaternary amine, examples are, but not limited to, hexadecyltrimethylammonium bromide (CTAB), tetrabutylammonium bromide (TBAB), 1-hexadecylpyridinium chloride, benzyldimethyltetradecylammonium chloride dehydrate (Zeph), trisdodecylmethylammonium chloride (TDMAC), tetrakis(dodecyl)ammonium bromide.

For the application of self-contained calcium specific and magnesium specific film sensor, the sensors described are attached to or immobilized in a solid matrix. The sensor formulation is then disposed as a film on the substrate. It is to be appreciated that the polymeric material used to produce the sensor film matrix may affect detection properties such as sensitivity, selectivity, and detection limit.

Suitable polymers, which may be used as polymer supports in accordance with the present disclosure, include hydrogels. As defined herein, a “hydrogel” is a three dimensional network of hydrophilic polymers which have been tied together to form water-swellable but water insoluble structures. The hydrogels could be synthesized via any polymerization method known in the art, such as radiation, free radical, chemical cross-linking, grafting from any suitable monomeric constituents. The polymers used in this invention are well known to those skilled in the art; however, an important aspect is that the film must be water-swellable or/and porous. Enhanced water swelling ability and porosity ensures rapid response. The polymer also should be compatible with analyte-specific reagents and auxiliary reagents (buffer, surfactants, plastizer, etc.) to maximize the binding sites offered per unit sensor film. Thus, a preferable hydrogel used in this invention is poly(2-hydroxyethylmethacrylate) (pHEMA).

The hydrogel polymer matrix is dissolved in a suitable solvent including, but not limited to, 1-methoxy-2-propanol (PM), 2-methoxyethanol, 2-ethoxyethanol, 2-butoxyethanol, ethylene glycol, ethylene glycol diacetate, di(ethylene glycol) methyl ether (DM), ethylene glycol monopropyl ether. In one aspect, the concentration of the solvent in the solution containing the polymer is in the range from about 70% (weight) to about 90%. In another aspect, the solvent could be any mixture on the above-mentioned solvents. Co-solvent might also be used to help dissolve any ingredients other than polymer to make homogeneous sensor formulations, for example, ethanol, methanol, water, acetone, and isopropyl alcohol.

The sensor film described herein may be self-standing or it may be further disposed on a substrate by means of various coating methods. The support substrates include, but not limited to, glass, plastic, paper or metal. The sensor film maybe applied or disposed on the substrate using any techniques known to those skilled in the art, for example, painting, spraying, spin-coating, dipping, screen-printing, air-knife coating, curtain coating, extrusion coating, Micro Gravure Coating.

The drying temperature and time for the solid film should be high and long enough to dry the film well where no significant amount of solvent residual left into film. The solvent residual might affect the sensor performance, and/or gradually evaporate to affect the calibration of the sensor. Secondly, the drying temperature and time should be low and short to avoid any degradation and side reaction taking place during drying process. For example, the drying temperature could be from room temperature to 200° C. depending on the boiling point, volatility of the solvent used in the formulation and the nature of the formulation. More specifically, the drying temperature could be from 60° C. to 130° C. from 5 minutes to 20 minutes. Even more specifically, the drying temperature and time could be from 70° C. to 95° C. with 7.5 minutes to 10 minutes.

The solids concentration of the formulations used to be coated on the surface should be low. For example, solids in the range from about 15% to 30% by weight should be used so as to not adversely affect the thickness of the film and its optical properties. In one aspect, the dry film thickness ranges from about 1 micron to about 60 microns, in another aspect; the thickness of the film is in the range from about 2 microns to about 40 microns. Another embodiment is that the thickness of the film is in the range from about 10 microns to about 22 microns.

Contacting of the solid film sensor with the test sample may be carried out by any suitable mechanism or technique. Some examples by which contacting may occur include, but are not limited to, dipping a strip of the sensor in a test-sample solution, spotting a sensor film with a sample solution, or flowing a test sample through a testing device having a film sensor and the like.

The contact of the film sensor with the analyte containing sample triggers a set of reactions that allow for the estimation of the amount of analyte present. The estimation can be done visually using a color chart to obtain a qualitative or semi-quantitative assay value, or using a small instrument especially designed for this purpose which measures some optical property (absorbance, luminescence, reflection, etc.), either in equilibrium or in a kinetic way. In one aspect, the small instrument could have multiple wavelength light sources, such as three color (RGB LED), and the absorbance of the film can be acquired by an aligned photodiode.

After measuring the change in the optical property, preferable absorbance, the calcium and magnesium concentration in the sample can be determined by converting the change in the optical property to the calcium and magnesium concentration. This conversion may be carried out using a calibration cure. The calibration curve may be prepared by measuring changes in an optical property of a calcium and magnesium sensor after contacting with test samples of known calcium and magnesium concentrations. After the calibration curve is generated, the calcium concentration in an unknown test sample may be determined by using the calibration curve. In one aspect, the change in absorbance of the calcium and magnesium sensor after contacting with a test sample is directly proportional to the calcium and magnesium concentration.

Self-contained calcium specific film sensor include calcium specific analyte-reagent, buffer and sensitivity (dynamic range) modifier reagent. “Calcium-specific reagents” can be used in the solid film sensor in this invention. Well developed and widely utilized “chromogenic” dyes are preferably used for the calcium-specific film sensor. Some example of the chromogenic dyes that can be used include triphenymethane dyes, azo dyes, O,N-donating chelating dyes. Some specific examples of chromogenic dyes include, but not limited to, Murexide, Arsenazo I, Arsenazo III, Antipyrylazo III, Antipyrylazo-m-Cl, Dibromo-p-methyl-methylsulfonazo, Chlorophosphonazo III, DBC-chlorophosphonazo, Antipyrylazo-m-SO3H, Sulfonazo III, Dimethylsulfonazo III, Amino G acid Chlorophosphonazo, Acid blue 158, Shigailing, Glyoxal bis(2-hydroxyanil), 2,3,4-Trihydroxyacetophenone, Di-r-chloride antipyrinum. All of these dyes can be in acid form, hydrated form, salt forms.

The choice of the pH-modifier depends upon the nature of the chromogenic dyes. Moreover, the pH level provided from the pH-modifier is favored to be close to the pH value of the test samples. Thus, the response variation of the calcium specific film sensor resulted from different pH and alkalinity can be minimized. Herein we disclose some examples as to how the chromogenic dyes and pH-modifier may be selected.

In a film sensor, Chlorophosphonazo III only complexes Ca when the pH is lower than 6.0 and higher than 3, which makes the dye Ca specific in the film sensor. However, when the pH is between 6 to 10.5, Chlorophosphonazo III will response to both Ca and Mg with different signal (absorbance) intensity. Moreover, the Chlorophosphonazo III will respond to Ca with a different sensitivity when the pH is between 4 and 6. So to keep the samples tested at a constant pH level, pH-modifiers are necessary for Ca specific measurement. To use salt (buffer) as pH-modifier, the salts could be either commercial available, or made in situ. For example, potassium hydrogen phthalate (KHP) is a commercially available product; KHP is often used as a primary standard for acid-base titrations because it is solid and air-stable, making it easy to weigh accurately. It is also used as a primary standard for calibrating pH meters because, besides the properties just mentioned, its pH in solution is very stable. KHP is widely used as a buffering agent (in combination with hydrochloric acid (HCl) or sodium hydroxide (NaOH) depending on which side of pH 4.0 the buffer is to be).

Because Chlorophosphonazo III has two sulfonic groups, it can exchange cations (H⁺ for acid form, Na⁺ for salt form) with quaternary ammonium salt to form hydrophobic featured dye-ammonium ion pairs. Thus, the dye is immobilized into the solid film matrix. When contacting aqueous sample, the dye is not leachable.

As discussed above, by contacting of the film sensor with the analyte, any detectable physical and chemical property changes could be used to determine the analyte concentration. For optical property changes, examples are but not limited, absorbance and fluorescence. Absorbance is widely utilized. The absorbance can be acquired by spectrophotometer as an absorption spectrum, or can be acquired with miniaturized multi-wavelength or mono-wavelength absorbance detector. Specifically, the detector with a multi-wavelength light source can be used. Even more specifically, a three color LED (red 636 nm, green 530 nm, blue 465 nm) may be used as light source, and a photodiode as light detector was used. Plotting the absorbance change at three wavelengths (R, G, B or Red, Green, Blue, or 636 nm, 530 nm, 465 nm) against the concentration of calcium will generate the response (calibration curve). However, as well known in the skill of the art, the ratiometric approach circumvents many of the problems of the intensity-based methods, including signal variations due to inhomogeneous dye concentrations, fluctuations in source intensity or temperature, and coloring of the samples.

All the discoveries disclosed for self-contained calcium specific film sensor can also apply for the development of self-contained magnesium specific film sensor.

For the dye (analyte-reagent) can specifically or preferably respond to magnesium, o,o′-dihydroxyarylazo compounds are widely accepted and known as a metal indicator in chelatometry, especially for the EDTA titration of alkaline earth metals. It is know that the formation constants of magnesium with o,o′-dihydroxyarylazo dyes are always greater than that of calcium toward the dye, although the difference between the two is variable. Typical examples are Eriochrome Black T, Calcon carboxylic acid, Calgamite and Hydroxynaphthol blue. All of these Mg specific reagents can also be in acid, hydrated, and salt forms. Here is one example. The absorption spectra of these dyes at different stages of deprotonation are significantly different, which means to use the dye as magnesium sensor, a preferable pH range or pH point have to been chosen. Generally, these dyes, in the presence of alkali metals, the blue species of dyes (at basic pH conditions, specifically, at pH 10) turn to reddish in the aqueous solutions, and such color reactions are utilized in the photometry of metal ions or in the chelatometry as metal indictors.

### Magnesium Specific Formulation

2.0 g pHEMA was dissolved into DM/EP solvent mixture.

1.30 triethanolamine

TBAB (tetrabutylamonium bromide) 0.60 g

Erichrome Black T

The invention has been described in detail with particular reference to preferred embodiments thereof, but it will be understood that variations and modifications can be effected within the spirit and scope of the invention.

1. Method of treating a film sensor of the type including a combination of polymeric matrix film material, analyte indicator and solvent which combination has been treated via a first heat application to volatize most of said solvent and to solidify said analyte indicator to form said film sensor, the improvement comprising subjecting said film sensor to a second heat treatment to set the film and improve stability and shelf life.

2. Method as recited in claim 1 wherein said second heating comprises heating said film sensor at a temperature of about 50° C. to 150° C. for a period of about 1 minute to 3 days.

3. Method as recited in claim 2 wherein said second heating comprises heating said film sensor at a temperature of about 70° C. to 130° C. for a period of about 5 minutes to 2 days.

4. Method as recited in claim 3 wherein said second reheating comprises heating said film for a period of 5 minutes to 10 hours.

5. Method as recited in claim 1 wherein said film sensor is adapted for determining Ca concentration in an aqueous sample, said polymer matrix comprising poly(2-hydroxyethylmethacrylate) (pHEMA) hydrogel and said analyte indicator comprising chlorophosphonazo III.

6. Method as recited in claim 1 wherein said film sensor is adapted for determining total hardness in an aqueous sample, said polymer matrix comprising (pHEMA) hydrogel and said analyte indicator comprising methylthymol blue.

7. Method as recited in claim 1 wherein said film sensor is adapted for determining Mg concentration in an aqueous sample, said polymer matrix comprising (pHEMA) hydrogel and said analyte indicator comprising Eriochrome Black T.
